(19) United States
(12) Patent Application Publication
(10) Pub. No.: US 2010/0160928 A1
(43) Pub. Date: Jun. 24, 2010

(54) APPARATUS FOR USE IN GYNAECOLOGIC SURGERIES

(76) Inventors: José Guillermo Rodriguez NAVAS, Ilopango (SV); Edgar GARCIA, San Marcos (NI); Erick Edgardo GARCIA, Edmonton (CA); Daniel Alberto GARCIA, Edmonton (CA); Julio Cesar GARCIA, Edmonton (CA)

Correspondence Address: DAVIS & BUJOLD, P.L.L.C., 112 PLEASANT STREET, CONCORD, NH, 03301, US

(21) Appl. No.: 12/644,199

(22) Filed: Dec. 22, 2009

Related U.S. Application Data
(60) Provisional application No. 61/139,799, filed on Dec. 22, 2008.

(51) Int. Cl. A61B 17/42

(52) U.S. Cl. 606/119

(57) ABSTRACT

An apparatus for use in gynaecological operations includes a support and a mounting assembly for mounting the support to an operating table. A vertical member is supported in a vertical orientation by the support and a horizontal bar is supported in a substantially horizontal orientation by the vertical member. An annular vaginal plug is supported by and is axially movable along the horizontal bar. A plug locking assembly is provided for locking the vaginal plug in a selected axial position along the horizontal bar. A uterine driver may be introduced through the abdominal wall that has a handle and a uterus engagement tip with omni-directional movement in the released position. The engagement tip is connected to the uterine driver by a flexible member.

### FIELD

The present invention relates to an apparatus used to simplify gynaecologic surgeries, such as of the ovaries, fallopian tubes, uterus and vagina by performing one or more functions that would otherwise be performed by a surgeon.

### BACKGROUND

The two traditional approaches to performing surgery on the gynaecologic organs include vaginal and open surgery. Two new approaches include laparoscopic and robotic surgery. Some tools that are used include: U.S. Pat. No. 5,409,496 (Rowden) entitled “Uterine manipulator with locking mechanism”, U.S. Pat. No. 5,520,698 (Koh) entitled “Simplified total laparoscopic hysterectomy”, U.S. Pat. No. 5,540,700 (Rowden) entitled “Uterine manipulator”, U.S. Pat. No. 5,487,377 (Smith) entitled “Uterine manipulator and manipulator tip assembly”, U.S. Pat. No. 7,175,634 (Van Heerden) entitled “Uterine manipulator device”, U.S. Pat. No. 5,382,252 (Failla) entitled “Transvaginal uterine manipulator”, U.S. Pat. No. 4,022,208 (Valtchev) entitled “Gynecologic instrument”.

### SUMMARY

There is provided an apparatus for use in uterus removal operations which includes a support and a mounting assembly for mounting the support to an operating table. A vertical member is supported in a vertical orientation by the support and a horizontal bar is supported in a substantially horizontal orientation by the vertical member. An annular vaginal plug is supported by and is axially movable along the horizontal bar. A plug locking assembly is provided for locking the vaginal plug in a selected axial position along the horizontal bar.

With the apparatus, as described above, the supporting surgeon is no longer required to manipulate the uterus. As hereinafter will be further described, other functions performed by the supporting surgeon can also be eliminated by adding further functionality to the apparatus.

Even more beneficial results may be obtained when a remote end of the horizontal bar has a uterus engagement tip to engage a uterus of a patient. The addition of this feature helps the horizontal bar (uterine bar) to engage the uterus of the patient to support the uterus in a raised position to provide better access for the leading surgeon, and also to extract the uterus during a laparoscopic hysterectomy.

Even more beneficial results may be obtained when a vaginal expansion sleeve is supported by and axially movable along the horizontal bar. Preferably, the vaginal expansion sleeve has an annular neck portion that engages the horizontal bar and a cervix engagement portion that overlies a cervix of a uterus of a patient. The vaginal expansion sleeve expands the vagina to provide better access for the leading surgeon.

### BRIEF DESCRIPTION OF THE DRAWINGS

The drawings are for the purpose of illustration only and are not intended to in any way limit the scope of the claims to the particular embodiment or embodiments shown, wherein:

FIG. 1 is a perspective view of the apparatus for use in uterus removal operations.

FIG. 2 is an exploded perspective view of the apparatus of FIG. 1.

FIG. 3 is an exploded side elevation view in section of the apparatus of FIG. 1.

FIG. 4 is a top plan schematic of the apparatus installed on an operating table adjacent to a patient.

FIG. 5 is a top plan schematic of the apparatus engaging the uterus of a patient.

FIG. 6 is a top plan schematic of the apparatus and a uterine driver engaging the uterus of a patient.

FIG. 7 is a cross-sectional view of the vaginal expansion sleeve and the vaginal plug.

FIG. 8 is an exploded cross-sectional view of the uterine driver.

FIG. 9 is a cross-sectional view of the uterine driver.

### DETAILED DESCRIPTION OF THE PREFERRED EMBODIMENT

The apparatus 10 is designed to aid surgeons and improve gynaecological surgeries, such as the different surgical techniques of the ovaries, fallopian tubes, uterus, vagina, etc. There are two components to the apparatus. The first component is an automatic uterus elevator and vaginal enhancer and the second is a uterus driver. The apparatus system can be used and assembled in different positions to create the surgical conditions the surgeon needs in order to perform the planned surgery.

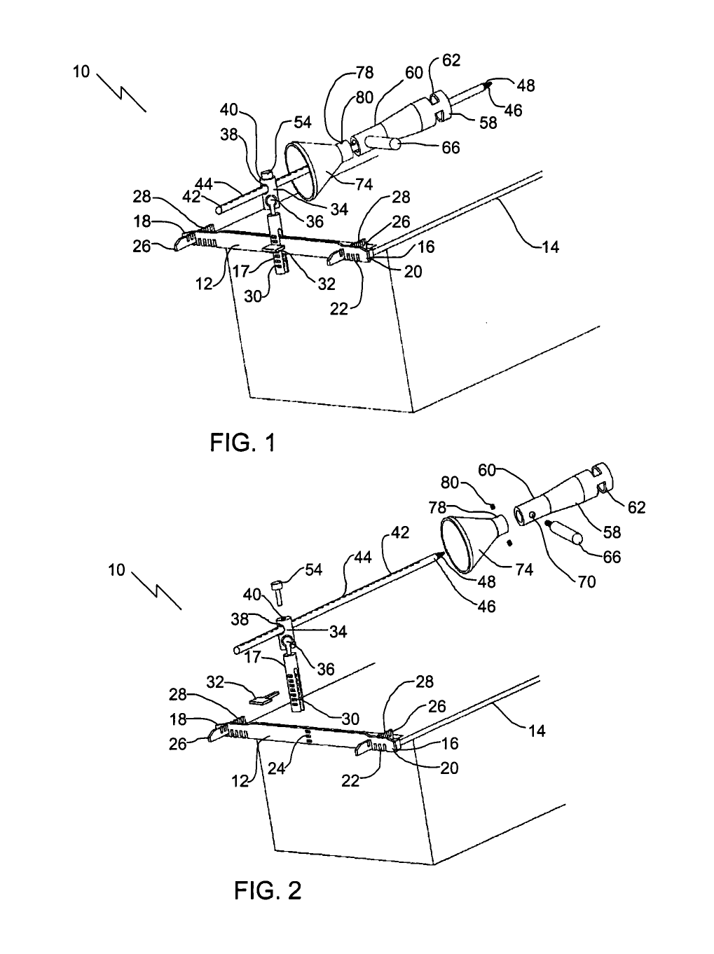

Referring to FIGS. 1 and 2, the automatic uterus elevator and vaginal enhancer includes a support 12 and a mounting mechanism 16 to mount onto an operating table 14. This keeps the first apparatus in a firm position without the need of an assistant. A vertical member 17 is supported in a vertical orientation by the support 12 and a horizontal bar 42 is supported in a substantially horizontal orientation by the vertical member 17. The vertical member 17 holds the horizontal bar 42, which has different roles, one of which being that its tip 48 may be specially designed to extract the uterus after hysterectomy. This reduces the surgical time during laparoscopic hysterectomy because the uterus can be extracted with relative ease compared with what happens with the current instruments in use that usually lose the uterus, or cannot extract the uterus, thereby wasting the time of the surgeon and of the surgical team that is tired because of the length of the operation. The horizontal bar 42 elevates the uterus and at the same time creates a better operating field that improves the exposure of the uterus, the fallopian tubes, the ovaries, the cardinal ligaments, the broad ligament, the round ligaments, the uterine vessels, and the vesicouterine plica. All of this is provided by the horizontal bar 42 without the need of any surgical assistant, which reduces the costs drastically. An additional advantage is the steady and controlled surgical field the surgeon gets with this apparatus without being concerned of the tiredness of the assistant because the apparatus is automatic and does not need any assistant once it is in place.

A vaginal expansion sleeve 58, which also acts as a uterus elevator is also supported by and axially movable mechanism along the horizontal bar 42. If the surgeon is only performing a surgery on the fallopian tubes or the ovaries, or repairing a vaginal enterocele, then the vaginal expansion sleeve 58 is introduced into the vagina—elevating all the internal gynaecologic organs while protecting the uterus from any trauma. This reproduces a comfortable surgical field. The surgeon can perform any technique with ease because all the structures are well exposed and readily accessible without needing to pull these organs with one's hands or other instruments that are only going to damage healthy tissues. At the same time, the surgical field is steady and under control, and there is little concern over losing the surgical field because the apparatus automatically retains itself. This reduces the number and skills of assistants the lead surgeon requires and allows the surgeon the use of both hands all of the time, except during the delicate maneuvers that are required by the technique. This can be applied to open or laparoscopic and robotic surgery.

An annular vaginal plug 74 is also supported and is axially movable along the horizontal bar 42. This vaginal plug 74 is specially designed for laparoscopic or robotic surgery, when it is necessary to open the vagina at any moment of the surgery. This vaginal plug avoids the leak of CO₂, and is designed in a way that fits perfectly with any size of vagina, regardless of its different anatomy.

When the surgeon is planning to perform laparoscopic or robotic hysterectomy, the uterus driver 82 is helpful. Uterus driver 82 is intended to be introduced through the anterior abdominal wall of the patient. This second component of the apparatus system is introduced through a port near the umbilicus. It has a uterus engagement tip 86 to secure the positioning of the uterus. The combination of the uterus elevator/vaginal enhancer 58 and the uterus driver 82 provides an improved surgical field and versatility to the lead surgeon. With this apparatus system the lead surgeon is given greater control of the surgical field. Now the surgeon can improve the exposure and accessibility of the structures. The surgeon does not need to depend on an assistant to perform the different maneuvers. With this apparatus system the surgeon can even handle complex cases that otherwise may not be possible in laparoscopic surgery. The uterus driver 82 and the vaginal sleeve 58 help control the uterine vessels; therefore, work over the vaginal formix and vesicouterine plica becomes easier. This apparatus system is intended to reduce the surgical time, reduces the anaesthesia time, simplifies the surgical technique, reduces costs, reduces complications, reduces unnecessary damage of normal tissue, reduces the number of assistants, makes the surgery very comfortable, and makes the surgical technique more standardized.

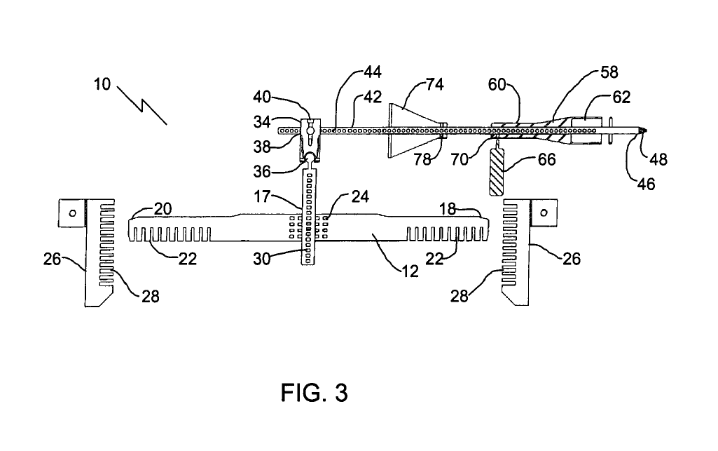

The preferred embodiment, an apparatus for use in uterus removal operations generally identified by reference numeral 10, will now be described with reference to FIG. 1 through 8.

Structure and Relationship of Parts:

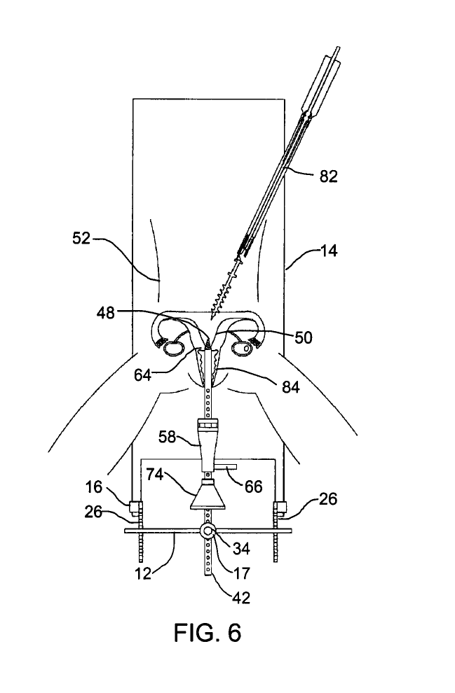

The components shown in FIGS. 1 and 2 may be referred to collectively as a uterus elevator and vaginal enhancer, which may then be used in combination with a uterine driver 82 as shown in FIGS. 6 and 8.

Referring to FIGS. 1 and 2, apparatus 10 includes a support 12. In the depicted embodiment, support 12 is mounted to an operating table 14 by a mounting assembly 16, and is used to support a vertical member 17. As shown, support 12 has opposed ends 18 and 20 with each opposed end 18 and 20 having engagement slots 22, and openings 24 positioned between opposed ends 18 and 20. Mounting assembly 16 as shown has a pair of spaced bars 26 with one or more engagement slots 28. Support 12 is mounted to operating table 14 by engaging selected engagements slots 22 in support 12 with selected engagement slots 28 in spaced bars 26 of mounting assembly 16. Vertical member 17 is supported in a vertical orientation by support 12 and has openings 30. A locking key 32 is inserted into one of the openings 30 to lock vertical member 17 at a selected height.

A receiver 34 is mounted at an upper extremity of vertical member 17 by a ball and socket engagement 36 that allows for omni-directional movement. Receiver 34 has a bore 38 with an intersecting engagement opening 40. A horizontal bar 42 is supported in a substantially horizontal orientation by bore 38 of receiver 34. Horizontal bar 42 has multiple openings 44. A remote end 46 of horizontal bar 42 has a uterus engagement tip 48 to engage a uterus 50 of a patient 52 as shown in FIG. 6. Referring to FIGS. 1 and 2, locking key 54 is provided that is inserted into one of the engagement openings 40 in receiver 34 and one of the openings 44 in horizontal bar 42 to lock horizontal bar 42 in a selected horizontal position.

Horizontal bar 42 may perform various roles, one of which being that its tip is specially designed to extract the uterus after hysterectomy. This reduces the surgical time during laparoscopic hysterectomy because the uterus is extracted with relative ease, contrary to what happens with the current instruments in use that usually lose the uterus, or cannot extract the uterus. Horizontal bar 42 can be used to elevate the uterus, and at the same time create a better operating field that improves the exposure of the uterus, the fallopian tubes, the ovaries, the cardinal ligaments, the broad ligament, the round ligaments, the uterine vessels, and the vesicouterine plica. All of this is provided by horizontal bar 42 without using a surgical assistant. This provides a steady and controlled surgical field to the surgeon while reducing the cost of an additional assistant, and also without concern for the tiredness of the assistant.

A vaginal expansion sleeve 58 is supported by and axially movable along horizontal bar 42. Vaginal expansion sleeve 58 has an annular neck portion 60 that engages horizontal bar 42 and a cervix engagement portion 62 that overlies a cervix 64 of uterus 50 in use. Vaginal expansion sleeve 58 may have a detachable handle 66 to facilitate positioning of cervix engagement portion 62 over cervix 64 of patient 52. Referring to FIG. 2, a sleeve locking assembly is provided that locks vaginal expansion sleeve 58 in a selected axial position along horizontal bar 42. The sleeve locking assembly includes an aperture 70 in vaginal expansion sleeve 58, and a locking body (not shown) that concurrently engages one of the openings 44 in horizontal bar 42 and aperture 70 in vaginal expansion sleeve 58.

If the surgeon is only performing a surgery on the fallopian tubes or the ovaries, or repairing a vaginal enterocele, then vaginal expansion sleeve 58 may be introduced into the vagina and elevate all the internal gynaecologic organs while protecting the uterus from any trauma. This reproduces a comfortable surgical field with the structures well exposed and readily accessible to the surgeon, and reduces the need to pull these organs with one's hands or other instruments that are only going to damage healthy tissues. At the same time, the surgical field provided is steady and controlled.

The risk of losing the surgical field is also reduced because apparatus 10 is automatic. This reduces the number of assistants the lead surgeon requires and reduces the demands on a surgeon's hands, at times allowing the surgeon the use of both hands, which would otherwise not be the case, except, for example, during the delicate maneuvers that are required by the technique. This can be applied to open or laparoscopic and robotic surgery.

An annular vaginal plug 74 is supported by, and axially movable along, horizontal bar 42 behind or over the vaginal expansion sleeve 58. A plug locking assembly 76 is provided for locking vaginal plug 74 in a selected axial position along horizontal bar 42. In one embodiment, a band 78 made from resilient material is used to lock vaginal plug 74. Vaginal plug 74 may be placed in front of vaginal expansion sleeve 58 on remote end 46 of horizontal bar 42 such that it covers vaginal expansion sleeve 58. In another embodiment, vaginal plug 74 may be locked using a pin connection that passes through a portion of plug 74 and engages horizontal bar 42. Other locking means will be apparent to those skilled in the art. Vaginal plug 74 is used to prevent leaks of CO₂, and is designed to fit a range of sizes of vaginas and different anatomies.

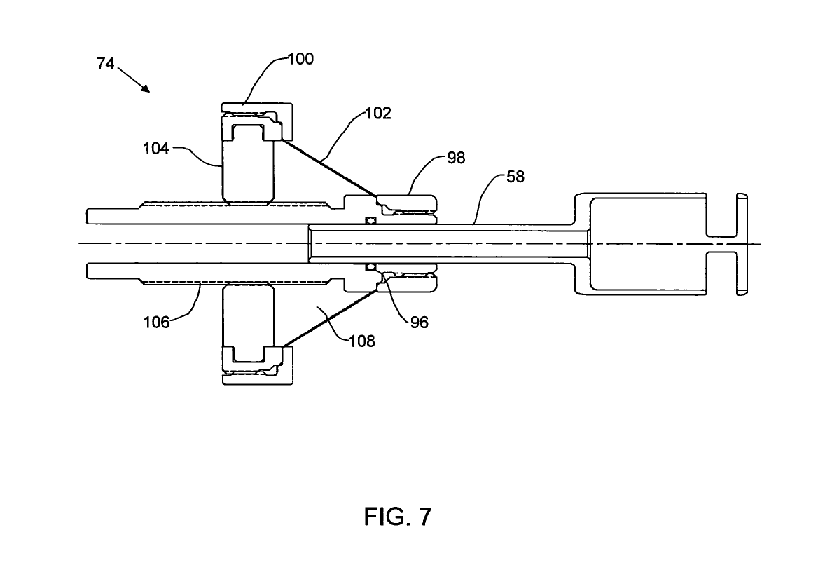

Referring to FIG. 7, an example of a vaginal plug 74 is shown. In the example shown, vaginal plug 74 overlies a rear portion of vaginal expansion sleeve 58. An o-ring seal 96 is provided to seal the connection and provide a friction fit. Vaginal plug 74 has a front portion 98 and a rear portion 100 connected by a cone-shaped diaphragm 102. The relative position of front portion 98 and rear portion 100 is adjustable by rotating threaded nut 104 connected to rear portion 100, which is mounted on a set of threads 106. As the relative position is changed, the slope of cone-shaped diaphragm 102 to adjust to different vagina sizes. Once properly positioned and sized, the cavity 108 within diaphragm 102 may be filled with cotton swabs (not shown).

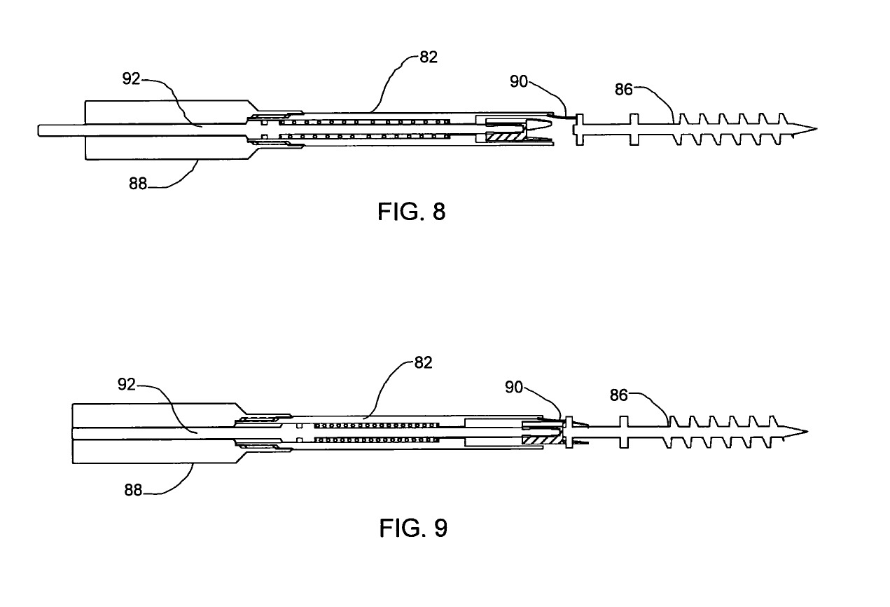

Referring to FIG. 8, a uterine driver 82 is provided that may be used to help position the uterus. Uterine driver 82 has a uterus engagement tip 86 that may have different shapes to anchor a uterus, such as a screw shape as shown, a nail or needle shapes etc. Uterine driver 82 also has a control handle 88 connected to engagement tip 86 by a flexible member, such as a cable 90 as shown, or multiple joints connected together. The flexible member may also be, for example, multiple joints or a series of pivoting, segmented members, or other known structures that provide flexibility. Control handle 88 has a spring-mounted inner shaft 92. When inner shaft 92 is retracted away from engagement tip 86 as shown in FIG. 8, engagement tip 86 is free to move omni-directionally while remaining attached to cable 90. When inner shaft 92 is extended as shown in FIG. 9, it engages with uterus engagement tip 86. Thus, when it is necessary to engage or release the uterus with engagement tip 86, inner shaft 92 is extended into engagement with engagement tip 86, and engagement tip 86 may then be manipulated by pushing or rotating control handle 88 until it properly engages the uterus. Once the uterus is properly engaged, inner shaft 92 is retracted to release the engagement between engagement tip 86 and control handle 88.

Operation:

There will now be given an example of how apparatus 10 may be used in an operation where the uterus is to be removed. It will be understood by those skilled in the art that apparatus 10 may be used to perform other gynaecological surgeries, such as surgeries involving the ovaries, fallopian tubes, uterus, vagina, etc.

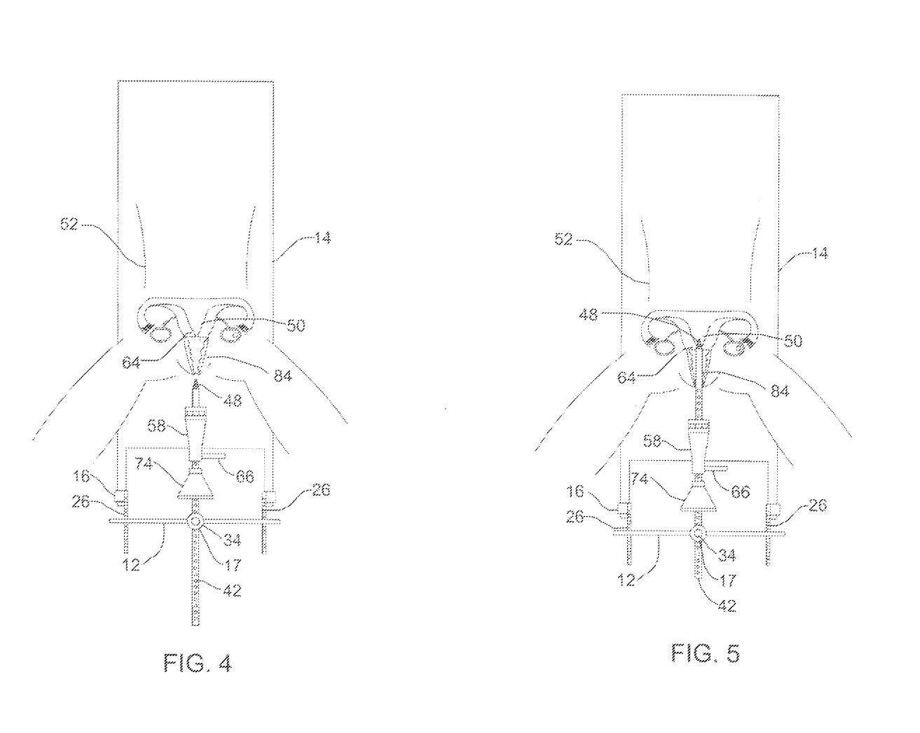

Referring to FIG. 1, apparatus 10 is assembled and attached to operating table as adjacent to patient 50, as depicted in FIG. 4. Referring to FIG. 5, uterus engagement tip 48 of horizontal bar 42 is screwed inside the cervix 64 of uterus 50, and pushes uterus 50 up to lift it into the desired position. Horizontal bar 42 is locked into position by locking key 54. Referring to FIG. 6, a uterine driver 82 is introduced into the abdomen through a port near the umbilicus of patient 52 and is screwed inside uterus 50.

The surgeon is now ready to start the operation comfortably using both hands to do all maneuvers all the time as in classical surgery, beginning with, for example, controlling the uterine vessels and separating the bladder from uterus 50, or with any modification of the technique that the surgeon prefers. Once the previous steps have been completed, referring to FIG. 7, vaginal expansion sleeve 58 is introduced to improve the visualization of vaginal formix and the vesicouterine plica for a comfortable colpotomy. Vaginal expansion sleeve 58 is kept in place with a pin that is inserted through aperture 78 and horizontal bar 42. Vaginal plug 74 is pushed inside the vagina 84 and is filled with a swab (not shown) to avoid any leak of CO₂ without being concerned about how wide or narrow the patient's vagina 84 is.

When the colpotomy is finished, horizontal bar 42 is disassembled from vertical member 17. Uterine driver 82 is used to push uterus 50 inside vagina 84 while horizontal bar 42 is pulled out. This allows a relatively easy, smooth and comfortable extraction of uterus 50, without delay. Once uterus 50 is out, vaginal plug 74 is introduced inside vagina 84 again to finish the operation. Once the operation is completely finished vaginal plug 74 is removed.

When the surgeon is planning to perform laparoscopic or robotic hysterectomy, uterine driver 82 may be used. Uterine driver 82 is preferably introduced through a port near the umbilicus. Uterus engagement tip 86 secures the positioning of uterus 50. The combination of the uterus elevator and vaginal enhancer shown in FIGS. 1 and 2 and the uterine driver shown in FIGS. 8 and 9 is used to provide a useful surgical field and versatility to the lead surgeon, which allows the lead surgeon increased control over the surgical field to expose the necessary structures and provide access to them. The need for the surgeon to depend on an assistant is reduced. It is anticipated that complex cases may also be performed, that would otherwise be difficult without laparoscopic surgery. As uterus engagement tip 48 of horizontal bar 42 and vaginal sleeve 58 help control the uterine vessels, work over the vaginal formix and vesicouterine plica is simplified. This apparatus system reduces the surgical time, reduces the anaesthesia time, simplifies the surgical technique, reduces costs, reduces complications, reduces unnecessary damage of normal tissue, reduces the number of assistants, makes the surgery very comfortable, and makes the surgical technique more standardized.

Advantages:

This instrument gives the surgeon any position needed to perform the operation efficiently, and without having to sacrifice one hand of the surgeon to separate the uterus as in current laparoscopic techniques. At the same time, the position of apparatus 10 is maintained, so there is no need of assistance from another surgeon between the legs of patient 52 to lift and maintain the position of uterus 50 as in current laparoscopic or robotic techniques. Apparatus 10 is thus able to speed the surgery and reduce the resources necessary because it assists in placing uterus 50 in the right position more easily.

The colpotomy becomes easier to perform because the uterus can be put in any position any time with relatively little effort, and the surgeon gets enough surgical field to do this and any other manoeuvre comfortably and quickly. The extraction of the uterus is also simplified because, while one instrument pushes the uterus into the vagina, the other is pulling it out. The need for a skilled assistant is reduced, because the main surgeon has control of the operation at all times and does not need to rely on the assistant to do important maneuvers.

For the above reasons, this apparatus shortens the surgical time, shortens the anaesthesia time, and reduces the amount of resources necessary to perform the operation, each of which reduces the cost of the operation. At the same time, it reduces complications, reduces unnecessary damage of normal tissue, and makes the surgical technique more standardized. This allows that more surgeons can perform this and other techniques increasing a substantial amount of patients that will benefit from it.

Variations:

The various components have been depicted as discrete elements. Other designs may provide the various components as permanently attached, either movable relative to each other or fixed, depending on the components involved. Furthermore, other designs may use different adjustable connections other than the slots and engagement holes depicted as the preferred embodiment. For example, geared connections, clamps, sliding clamps, etc. may be used as will be recognized by those skilled in the art.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from what is claimed.

What is claimed is:

1. An apparatus for use in gynaecological operations, comprising:
a support;
a mounting assembly for mounting the support to an operating table;
a vertical member supported in a vertical orientation by the support;
a horizontal bar supported in a substantially horizontal orientation by the vertical member;
an annular vaginal plug supported by and axially movable along the horizontal bar;
a plug lock for locking the vaginal plug in a selected axial position along the horizontal bar.

2. The apparatus of claim 1, wherein a remote end of the horizontal bar has a uterus engagement tip to engage a uterus of a patient.

3. The apparatus of claim 1, wherein a vaginal expansion sleeve is supported by and axially movable along the horizontal bar, the vaginal expansion sleeve having an annular neck portion that engages the horizontal bar and a cervix engagement portion that overlies a cervix of a uterus of a patient.

4. The apparatus of claim 3, further comprising a locking assembly for locking the vaginal expansion sleeve in a selected axial position along the horizontal bar.

5. The apparatus of claim 3, wherein the vaginal expansion sleeve has a detachable handle to facilitate positioning of the cervix engagement portion over the cervix of the patient.

6. The apparatus of claim 1, wherein the support has one or more openings and the vertical member has one or more openings, a locking key being inserted into a selected one of the openings in the support and a selected one of the openings in the vertical member to lock the vertical member at a selected height.

7. The apparatus of claim 1, wherein the vertical member has a receiver into which the horizontal bar is received, the receiver having one or more openings and the horizontal bar having one or more openings, a locking key being inserted into a selected one of the openings in the receiver and a selected one of the openings in the horizontal bar to lock the horizontal bar in a selected horizontal position.

8. The apparatus of claim 1, wherein the receiver is mounted by a ball and socket engagement at an upper extremity of the vertical member.

9. The apparatus of claim 1, wherein the mounting assembly has a pair of spaced bars with one or more engagement slots, and the support has opposed ends with each of the opposed ends having one or more complementary engagement slots, the support being mounted to the operating table by engaging selected engagements slots in the support with selected engagement slots in the spaced bars of the mounting assembly.

10. The apparatus of claim 1, wherein the plug locking assembly comprises a plurality of openings in the horizontal bar, an aperture in the vaginal plug, and at least one locking body that concurrently engages a selected one of the openings in the horizontal bar and the aperture in the vaginal plug.

11. The apparatus of claim 1, further comprising a uterine driver having a uterus engagement tip for engaging a uterus of a patient, the uterine driver being separate and distinct from the horizontal bar.

12. The apparatus of claim 11, wherein the uterine driver comprises a control handle that selectively engages the engagement tip.

13. An apparatus for use in gynaecological operations, comprising:
a support;
a mounting assembly for mounting the support to an operating table;
a vertical member supported in a vertical orientation by the support;
a horizontal bar supported in a substantially horizontal orientation by the vertical member, a remote end of the horizontal bar has a uterus engagement tip to engage a uterus of a patient;
a vaginal expansion sleeve is supported by and axially movable along the horizontal bar, the vaginal expansion sleeve having an annular neck portion that engages the horizontal bar and a cervix engagement portion that overlies a cervix of the uterus of the patient;
means for maintaining the vaginal expansion sleeve in a selected axial position along the horizontal bar;
an annular vaginal plug supported by and axially movable along the horizontal bar; and
a plug locking assembly for locking the vaginal plug in a selected axial position along the horizontal bar.

14. The apparatus of claim 13, wherein the vaginal expansion sleeve has a detachable handle to facilitate positioning of the cervix engagement portion over the cervix of the patient.

15. The apparatus of claim 13, wherein the support has one or more openings and the vertical member has one or more openings, a locking key being inserted into a selected one of the openings in the support and a selected one of the openings in the vertical member to lock the vertical member at a selected height.

16. The apparatus of claim 13, wherein the vertical member has a receiver into which the horizontal bar is received, the receiver having one or more openings and the horizontal bar having one or more openings, a locking key being inserted into a selected one of the openings in the receiver and a selected one of the openings in the horizontal bar to lock the horizontal bar in a selected horizontal position.

17. The apparatus of claim 16, wherein the receiver is mounted by a ball and socket engagement at an upper extremity of the vertical member.

18. The apparatus of claim 13, wherein the mounting assembly has a pair of spaced bars with one or more engagement slots, and the support has opposed ends with each of the opposed ends having one or more complementary engagement slots, the support being mounted to the operating table by engaging selected engagements slots in the support with selected engagement slots in the spaced bars of the mounting assembly.

19. The apparatus of claim 13, wherein the plug locking assembly comprises a plurality of openings in the horizontal bar, an aperture in the vaginal plug, and at least one locking body that concurrently engages a selected one of the openings in the horizontal bar and the aperture in the vaginal plug.

20. The apparatus of claim 13, further comprising a uterine driver having a uterus engagement tip for engaging a uterus of a patient, the uterine driver being separate and distinct from the horizontal bar.

21. The apparatus of claim 20, wherein the uterine driver comprises a control handle that selectively engages and releases the engagement tip, in the release position the engagement tip being capable of omni-directional movement.

22. The apparatus of claim 21, wherein the engagement tip is connected to the uterine driver by a flexible member.

23. An apparatus for use in gynaecological operations, comprising:
a support having opposed ends with each of the opposed ends having one or more engagement slots, with one or more openings being positioned between the opposed ends;
a mounting assembly for mounting the support to an operating table, the mounting assembly having a pair of spaced bars with one or more engagement slots, the support being mounted to the operating table by engaging selected engagements slots in the support with selected engagement slots in the spaced bars of the mounting assembly;
a vertical member supported in a vertical orientation by the support, the vertical member having one or more openings;
a locking key inserted into a selected one of the openings in the support and a selected one of the openings in the vertical member to lock the vertical member at a selected height;
a receiver mounted for omni-directional movement by a ball and socket engagement at an upper extremity of the vertical member, the receiver having a bore with an intersecting engagement opening;
a horizontal bar supported in a substantially horizontal orientation by the bore of the receiver at the upper extremity of the vertical member, a remote end of the horizontal bar having a uterus engagement tip to engage a uterus of a patient, the horizontal bar having one or more openings;
a uterine driver having a uterus engagement tip for engaging a uterus of a patient and a control handle, the uterus engagement tip being connected to the uterine driver by a flexible member, the control handle selectively engaging and releasing the engagement tip, in the release position the engagement tip being capable of omni-directional movement;
a locking key inserted into the engagement opening in the receiver and a selected one of the openings in the horizontal bar to lock the horizontal bar in a selected horizontal position;
a vaginal expansion sleeve is supported by and axially movable along the horizontal bar, the vaginal expansion sleeve having an annular neck portion that engages the horizontal bar and a cervix engagement portion that overlies a cervix of the uterus of the patient, the vaginal expansion sleeve has a detachable handle to facilitate positioning of the cervix engagement portion over the cervix of the patient;
a sleeve locking assembly for locking the vaginal expansion sleeve in a selected axial position along the horizontal bar comprising an aperture in the vaginal expansion sleeve, and at least one locking body that concurrently engages a selected one of the openings in the horizontal bar and the aperture in the vaginal expansion sleeve;
an annular vaginal plug supported by and axially movable along the horizontal bar; and
a plug locking assembly for locking the vaginal plug in a selected axial position along the horizontal bar comprising an aperture in the vaginal plug, and at least one locking body that concurrently engages a selected one of the openings in the horizontal bar and the aperture in the vaginal plug.
